(19) United States
(12) Patent Application Publication
(10) Pub. No.: US 2005/0283040 A1
(43) Pub. Date: Dec. 22, 2005

(54) Incontinence sling

(75) Inventor: E. Skott Greenhalgh, Wyndmoor, PA (US)

Correspondence Address: SYNNESTVEDT & LECHNER, LLP, 2600 ARAMARK TOWER, 1101 MARKET STREET, PHILADELPHIA, PA, 191072950, US

(73) Assignee: Secant Medical, LLC, Perkasie, PA (US)

(21) Appl. No.: 11/151,980

(22) Filed: Jun. 14, 2005

Related U.S. Application Data
(60) Provisional application No. 60/580,171, filed on Jun. 16, 2004.
(60) Provisional application No. 60/606,977, filed on Sep. 3, 2004.

(51) Int. Cl. A61F 2/02

(52) U.S. Cl. 600/30

(57) ABSTRACT

A sling for controlling urinary incontinence is disclosed. The sling is formed from a tube having substantially lengthwise inextensible end portions and an elastically lengthwise extensible intermediate portion between the end portions. The intermediate portion has a U-shape which cradles the urethra. The end portions extend through the abdominal tissue to anchor the intermediate portion in position. The sling places the urethra under a transverse compressive load to hold it closed and prevent inadvertent urination. The tube is formed from interlaced filamentary members. Interlacing may be by warp knitting, weaving using a leno weave or braiding using a tri-axial braid structure. The end portions have a rough texture to facilitate anchoring in the tissue of the abdominal wall. The intermediate portion is smooth and soft to prevent tissue erosion.

### FIELD OF THE INVENTION

This invention relates to a device implantable within the abdomen to treat urinary incontinence.

### BACKGROUND OF THE INVENTION

In the United States, more than 13 million people suffer from the effects of urinary incontinence. Although significant numbers of men are afflicted, women suffer-from this disorder in disproportionate and overwhelming numbers.

Some factors which lead to incontinence in women include the effects of childbirth, hysterectomies, urinary tract infections, relaxation of the pelvic muscles and the thinning of urethral tissue associated with hormone reduction during menopause. These factors contribute to a weakening of the urinary sphincter muscles (located beneath the bladder surrounding the urethra) which may lead to “stress incontinence”, “urge incontinence” or a mixture of both types of incontinence. Stress incontinence is associated with the involuntary leakage of urine due to increased pressure on the bladder occasioned by such mundane actions such as coughing, sneezing, laughing, bending or lifting heavy objects. Urge incontinence occurs when one has the need to urinate but is unable to prevent leakage until proper facilities are reached.

FIG. 1 shows a sling 10 presently used for treating urinary incontinence. Sling 10 comprises an elongated flat strap or tape of material that is surgically implanted within the person's abdomen 12 between the vagina 14 and the urethra 16. The sling 10 has a U-shaped portion 18 that cradles and supports the urethra. Extension portions 20 attached to the U-shaped portion extend through the abdomen away from the vagina and anchor the sling within the abdomen. The extension portions may be attached to the pubic bone 22 as shown or may merely be anchored within the tissue of the abdomen. The U-shaped portion 18 places the urethra under compression and constricts it so that it takes some muscular effort to exert pressure on the bladder and force urine from it through the constricted urethra. The tension in the sling determines the amount of muscular effort required to effect urination.

There are several disadvantages to this device. It is not always possible to establish the proper sling tension. Too much tension means that it will be difficult, if not impossible, for the person to urinate using muscular contractions that compress the bladder. Too little tension means that the disorder remains uncorrected despite the person having undergone the procedure. It is, furthermore, difficult to adjust the tension once the sling is implanted. Adjustment is desirable because over time, the muscles and other tissue of the abdomen change in their compliance, strength and tone, and what may be sufficient tension at one point in time may be too much or too little later, leading to problems which must again be addressed by invasive surgery. Probably the worst problem associated with slings currently in use is known as “erosion”, whereby, in response to normal movement and pressure of the abdominal muscles, the edge of the sling, being relatively sharp, cuts through the abdominal tissue and enters the urethra. Erosion is indicated by burning pain during urination.

There is clearly a need for an improved sling for the treatment of urinary incontinence that does not suffer the disadvantages of present treatment devices.

### SUMMARY OF THE INVENTION

The invention concerns a urinary incontinence sling positionable in the abdomen between the urethra and the vagina to compress the urethra. In one embodiment, the sling comprises an elongated tube having opposite end portions and an intermediate portion positioned between the end portions. The intermediate portion is bendable to form a substantially U-shaped cradle positionable adjacent to the urethra. The end portions are positionable so as to extend through the abdomen in a direction away from the vagina. The end portions are in anchoring engagement with living tissue forming the abdomen. The intermediate portion compresses the urethra with a predetermined force.

The tube has a cross sectional shape which may be circular, oval or elliptical for example. Preferably, the tube comprises a plurality of interlaced filamentary members. The filamentary members may be interlaced by knitting, weaving or braiding. The end portions are substantially inextensible, and the intermediate portion is lengthwise elastically extensible.

In another embodiment, an elastically expandable and contractible body is positioned within the intermediate portion of the tube. The body has a predetermined internal pressure. The pressure substantially determines the compressive force exerted on the urethra by the tube.

The tube may comprise a pouch positioned at the intermediate portion of the tube. The pouch is defined by closing the tube at two locations in spaced apart relation to one another. The pouch is used to contain the expandable and contractable body within the intermediate portion.

In another embodiment, the urinary incontinence sling comprises elongated end portions oppositely disposed and an intermediate portion positioned between the end portions. The intermediate portion is bendable to form a substantially U-shaped cradle positionable adjacent to the urethra. The end portions are substantially inextensible and are positionable so as to extend through the abdomen in a direction away from the vagina. The end portions are in anchoring engagement with living tissue forming the abdomen. The intermediate portion compresses the urethra with a predetermined force. In this embodiment, the intermediate portion is preferably lengthwise elastically extensible. The end portions preferably comprise elongated straps and the intermediate portion comprises a tube. An elastically expandable and contractible body may be positioned within the tube that comprises the intermediate portion. The body has a predetermined internal pressure that substantially determines the compressive force exerted on the urethra by the intermediate portion.

### BRIEF DESCRIPTION OF THE DRAWINGS

FIG. 1 is a partial perspective anatomical view of the female abdominal region illustrating use of a prior art incontinence sling;

FIG. 2 is a perspective view of a sling according to the invention;

FIG. 3 is a side view of an embodiment of a sling according to the invention;

FIG. 4 is a partial sectional view of an abdomen in the medial plane;

FIG. 5 is a partial perspective view on an enlarged scale taken from within circle 5 of FIG. 4;

FIG. 6 is a perspective view of an embodiment of a sling according to the invention;

FIG. 7 is a perspective view showing the sling of FIG. 6 implanted within an abdomen;

FIG. 8 is a perspective view of an embodiment of a sling according to the invention;

FIG. 9 is a side view of an embodiment of a sling according to the invention and a tool for implanting the sling within an abdomen; and

FIGS. 10-12 illustrate steps in the use of the tool shown in FIG. 9 to implant the sling in an abdomen.

### DETAILED DESCRIPTION OF THE EMBODIMENTS

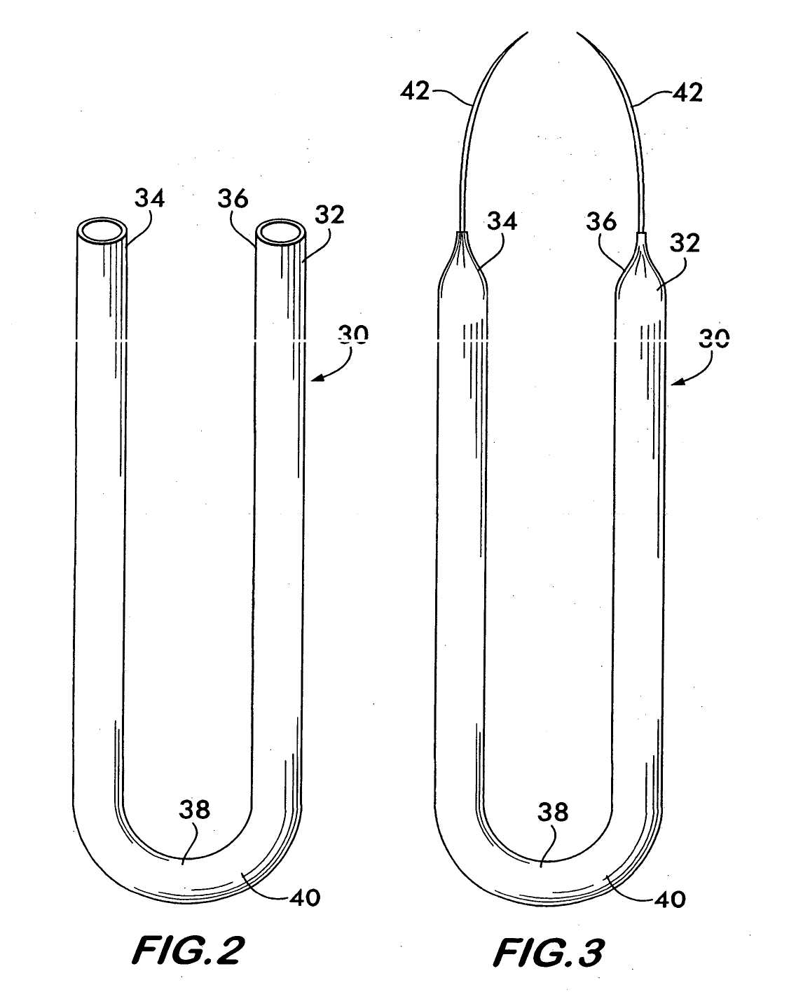

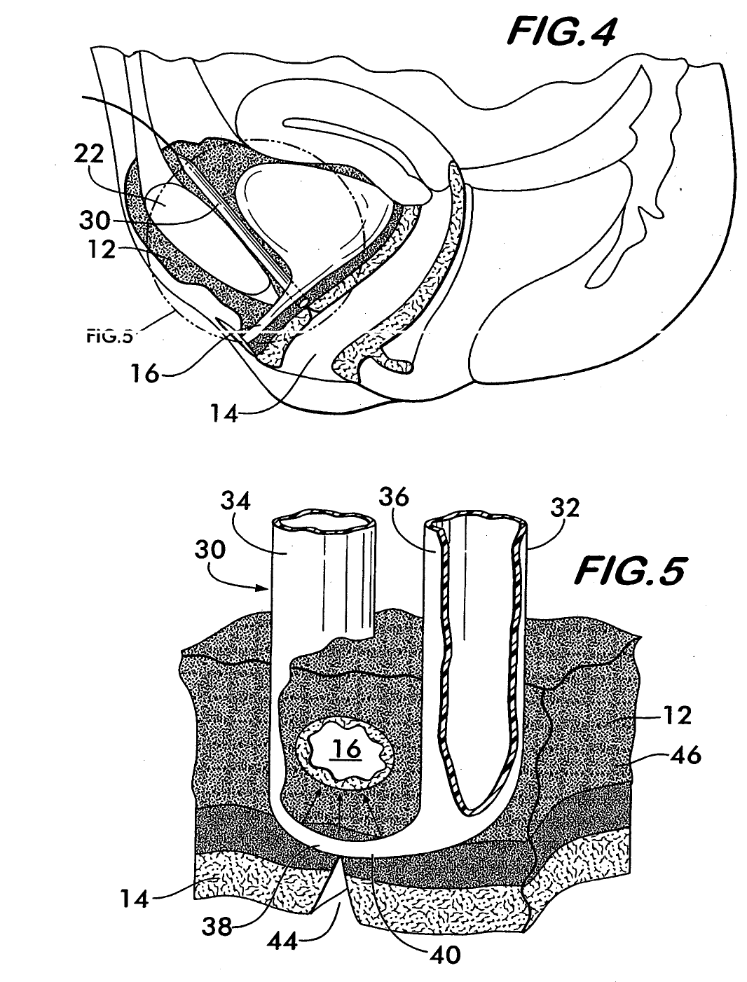

FIG. 2 shows an incontinence sling 30 according to the invention. Sling 30 comprises an elongated flexible tube 32 having opposite end portions 34 and 36. An intermediate portion 38 is positioned between end portions 34 and 36. The intermediate portion 38 is bendable to form a substantially U-shaped cradle 40 positionable adjacent to the urethra 16 as shown in FIGS. 4 and 5. End portions 34 and 36 are positionable so as to extend through the abdomen 12 away from the vagina 14. The end portions 34 and 36 anchor the sling 30 in position within the abdomen as described in detail below.

End portions 34 and 36 are substantially inextensible lengthwise, especially in comparison with center portion 38, which is lengthwise elastically extensible. By varying the longitudinal stiffness as a function of position along the sling 30 it is possible to achieve better control over the transverse compressive force applied to the urethra and thereby avoid the aforementioned problems associated with too little or too much sling tension. Furthermore, because they are substantially inextensible, the end portions 34 and 36, which anchor the sling 30 within the abdomen, are less susceptible to the effects of “sling recoil” as described in detail below.

In a preferred embodiment, sling 30 is formed from warp knitted polypropylene monofilaments.

Polypropylene is preferred because it is bio-compatible, provokes a healing response from living tissue and has a history of success as a material implantable within the human body. Other polymers such as nylon, polyester and polytetrafluoroethylene are also feasible as are bio-absorbable materials such as polyglycolic acid, polylactic acid, PEA, PEUR, PEG and PLLA.

Warp knitting is preferred because it provides a filamentary mesh structure that yields a substantially lengthwise inextensible tube 32, advantageous for reasons discussed in detail below. The axial stiffness that governs the lengthwise extensibility of the various portions may be controlled by increasing or decreasing the number of warp yarns in a portion, by introducing warp yarns having greater or lesser diameter, by making the warp yarns from materials having greater or lesser moduli of elasticity, or by a combination of any of these techniques. The substantially inextensible end portions 34 and 36 preferably have an extensibility between about 10% to about 20% per unit load, with about 15% extensibility per unit load being most preferred.

Tube 32 may also be woven, the leno weave being preferred for woven embodiments because it too produces an axially inextensible structure. Braiding is also feasible, with the triaxial braid providing the desired control over lengthwise elongation of the tube. The tube may also be a substantially continuous membrane, preferably formed of expanded polytetrafluoroethylene.

The tube 32 preferably has a round cross-section with a diameter of about 7 mm. Other cross-sectional shapes, such as oval, ellipsoidal or polygonal are of course feasible. The width of tube 32 is about 10 mm when flattened in contact with the abdominal tissue as shown in FIG. 5. The tube 32 is about 12 inches long before implanting.

Implanting of sling 30 is described with reference to FIGS. 3-5. As shown in FIG. 3, curved tunneling needles 42 are attached to end portions 34 and 36 of tube 32. Tunneling needles 42 are used to penetrate the abdominal tissue and draw the sling 30 through the abdomen 12 on opposite sided of the urethra 16 as shown in FIG. 4. As shown in detail in FIG. 5, an incision 44 is made in the vagina 14 adjacent to the urethra 16. Needles 42 are inserted through the incision 44 to pass on opposite sides of the urethra 16. The needles 42 are forced through the abdomen tissue 46 and exit through the abdominal anterior surface where they are drawn out of-the patient, thereby drawing the sling 30 through the incision 44, around the urethra 16 and through the abdominal tissue 46. The appropriate tension is placed on the sling and the end portions 34 and 36 are trimmed so that they remain beneath the surface of the skin of the abdomen.

Sling 30 is anchored in position by end portions 34 and 36. Anchoring may be effected in various ways, for example, by the intergrowth of abdominal tissue 46 through the end portions 34 and 36. For such anchoring, it advantageous to provide end portions 34 and 36 with appropriate material properties, surface texture and porosity that favors tissue ingrowth so that a strong anchoring of the sling is quickly achieved. Materials such as polypropylene are used in the end portions because polypropylene is known to provoke an aggressive healing response in living tissue. The surface texture of the end portions may be enhanced by the addition of a knap or by annealing the surface by the application of heat, or by using a particular stitch or multifilament yarn which gives a desired roughness. Furthermore, the porosity of the end portions 34 and 36 may be tailored to have interstitial spaces sized to promote tissue ingrowth. Porosity may be controlled by weave or stitch density as well as by varying the size of the filaments and their type, i.e., multifilaments, monofilaments and texturized filaments may be combined to yield a desired effect. The healing response may also be encouraged by the use of coatings on the end portions 34 and 36 such as thrombin and collagen.

It is advantageous that cradle 40 have a smooth, soft surface that helps to mitigate erosion. To that end, the intermediate portion 38 may have different characteristics from the end portions 34 and 36, for example, the density of the weave, knit or braid may be greater to better distribute the forces applied to the urethra 16. Alternately, it may be advantageous to form intermediate portion 38 from a lower density material with a correspondingly softer surface.

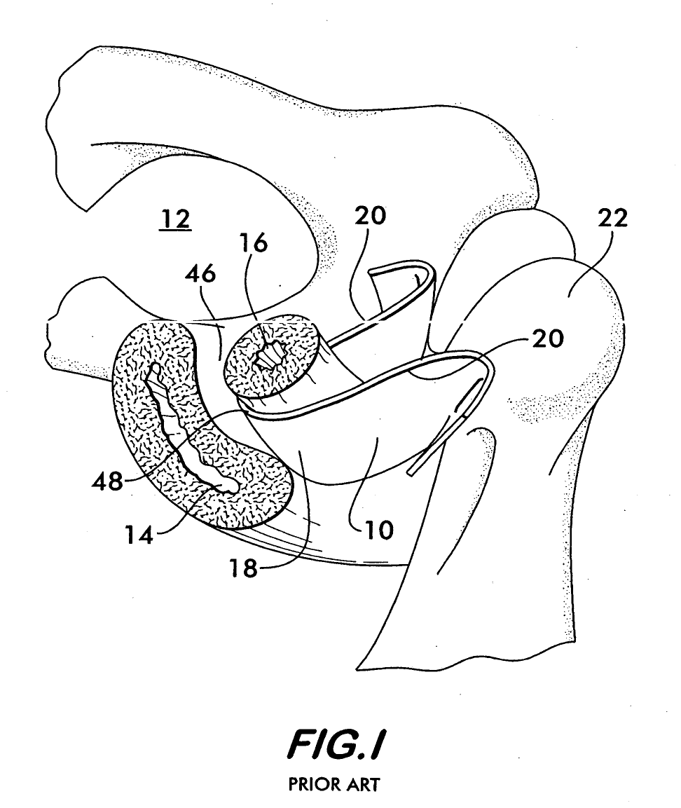

Two advantages of the sling 30 according to the invention may be explained with reference to FIGS. 1 and 5. The first advantage is how the sling 30 mitigates or eliminates the detrimental effect of erosion. As explained above (see FIG. 1), erosion occurs as a result of normal body movement and describes a condition whereby the edge 48 of the U-shaped portion 18 of prior art incontinence sling 10 cuts through the abdominal tissue 46 and enters the urethra 16. This happens largely because prior art slings are flat strips with two opposite edges that are relatively stiff and sharp as compared with the tissue in which they reside. If the strip forming the prior art sling is twisted when it is implanted, the risk of erosion is greatly increased. However, as shown in FIG. 5, sling 30 according to the invention is a tube and not a flat strip, and therefore cannot present an edge to any portion of the tissue between the vagina 14 and the urethra 16 regardless of its orientation or if it is twisted. Tubular slings 30 according to the invention are far less likely to cause erosion than prior art flat strip slings because they have no sharp edge.

The second advantage of the sling 30 is provided by the manner of interlacing the filamentary members comprising the tube 32 which emphasizes control over the axial stiffness of the tube. As explained above for the sling 30 according to the invention, the particular knit, weave or braid is chosen to produce a substantially lengthwise inextensible tube (i.e., little to no stretch under tensile load). The advantage of an inextensible tube is that, once set during implantation, the pressure exerted on the urethra 16 by the sling will not change significantly due to “recoil” of the sling.

Recoil occurs most acutely with prior art slings 10, shown in FIG. 1, that have significant axial flexibility. As the sling 10 is drawn through the abdominal tissue during implantation, it encounters significant resistance in the form of surface drag. The drag applies a tensile load to the sling, notably the extension portions 20, which, if flexible, will stretch lengthwise, placing the sling in a state of elastic tension within the abdominal tissue. Further tension (and stretching) may be applied to the extension portions 20 as the sling 10 is adjusted to apply pressure to the urethra 16. Because the extension portions 20 have stretched, they are under tension and therefore they constantly try to shrink back to their nominal length and relieve the tension. This shrinkage occurs during the healing process before the extension portions are anchored and the result is an increase in the force applied to the urethra as the U-shaped portion 18 is drawn toward the extension portions 20. The increased force on the urethra 16 caused by the recoil of the sling may mean that too much pressure is exerted by the sling 10, making it difficult to impossible to urinate by exerting muscle pressure on the bladder.

Sling 30, being substantially lengthwise 20 inextensible, avoids the problems associated with sling recoil. As they are drawn through the abdominal tissue 46, the end portions 34 and 36 of sling 30 do not stretch significantly and thus will not recoil and change the pressure on the urethra after implanting of the sling. Furthermore, because there is so little elastic tension within the end portions 34 and 36 as compared with the center portion 38 where the elasticity is concentrated, the pressure on the urethra may be established more precisely and reliably, as it is not necessary to account for the elasticity of the entire sling, only the more limited central portion 38. Thus, by concentrating the elasticity of the sling in the center portion 38 (and not in the end portions 34 and 36) the problem of changing sling tension due to recoil is substantially reduced or eliminated.

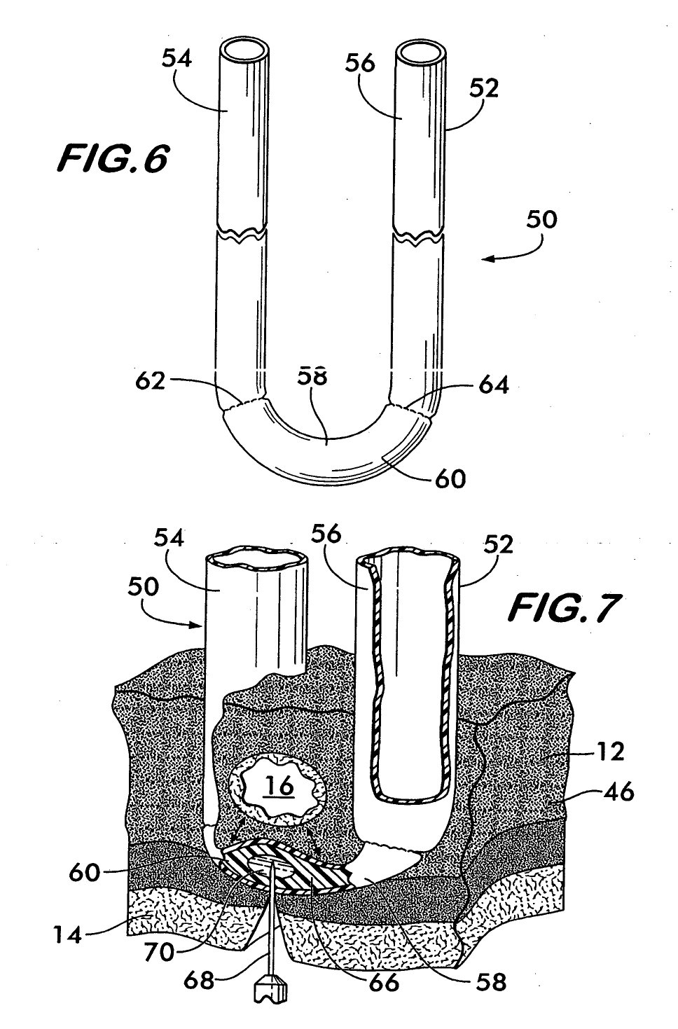

An alternate embodiment of a sling 50 allowing for adjustability of the pressure on the urethra is shown in FIG. 6. Sling 50 is formed from a tube 52 having end portions 54 and 56 and an intermediate portion 58 as described above. A pouch 60 is positioned in the intermediate portion 58. Pouch 60 may be formed in any number of ways and preferably by sealing the tube closed at two points 62 and 64 in spaced apart relation to one another. Sealing may be by heat fusion, sewing, interknitting, interweaving or interbraiding of the tube. Once the sling 50 is implanted, as shown in FIG. 7, pouch 60 is filled with a compound such as silicone that is bio-compatible. The silicone body 66 is injected through a needle 68 in liquid form and then cures to form an elastic solid. While body 66 is curing a gas is injected into it to form a chamber 70 within the body 66. After the body 66 is cured, the gas is replaced by a liquid. The body 66 with chamber 70 then forms an expandable and contractible body like a balloon within pouch 60 within which a fluid is maintained. If it becomes necessary to later change the pressure exerted by the sling 50 on the urethra 16, this may be accomplished by inserting a needle into the chamber 70 and pumping in or removing fluid to increase or decrease the volume of the body 66.

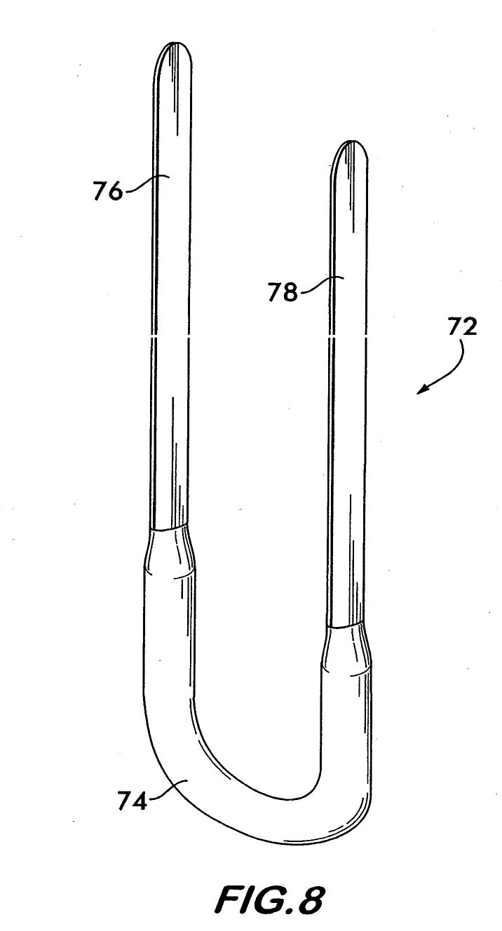

FIG. 8 illustrates another embodiment of a sling 72 according to the invention. Sling 72 has a tubular intermediate portion 74. From each end of intermediate portion 74 straps 76 and 78 extend. This sling embodiment is formed so as to be lengthwise inextensible so as to mitigate sling recoil. Straps 76 and 78 are relatively thin so as to better promote tissue ingrowth and anchoring during healing. The intermediate portion 74 has the tubular configuration so as to avoid erosion.

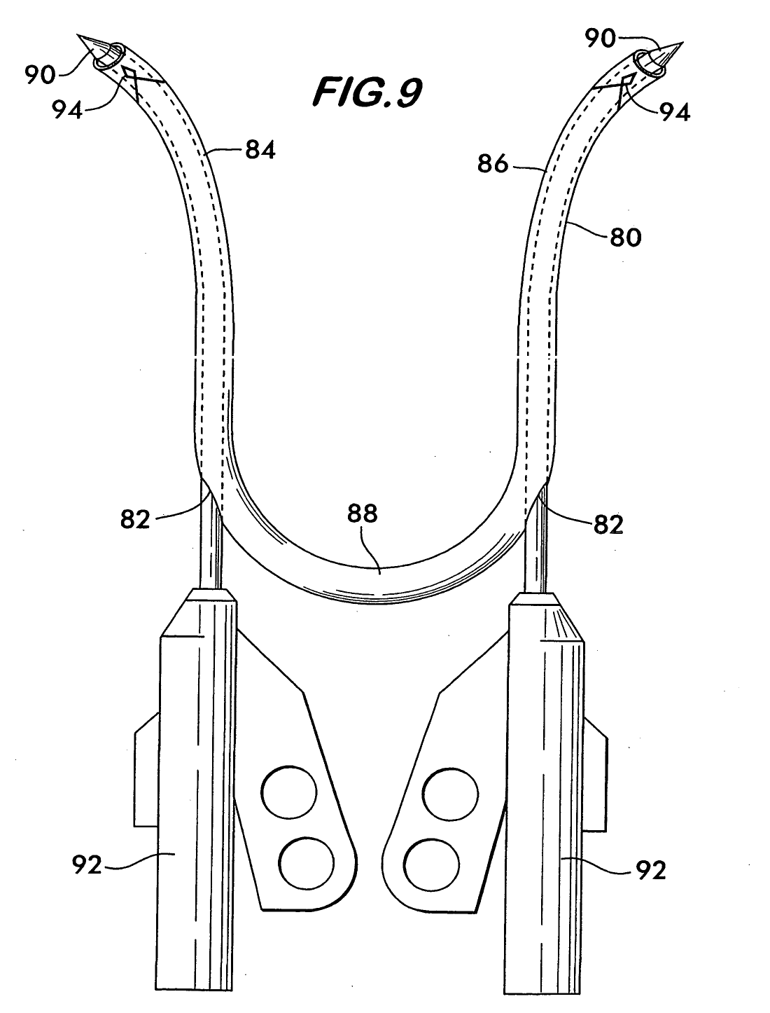

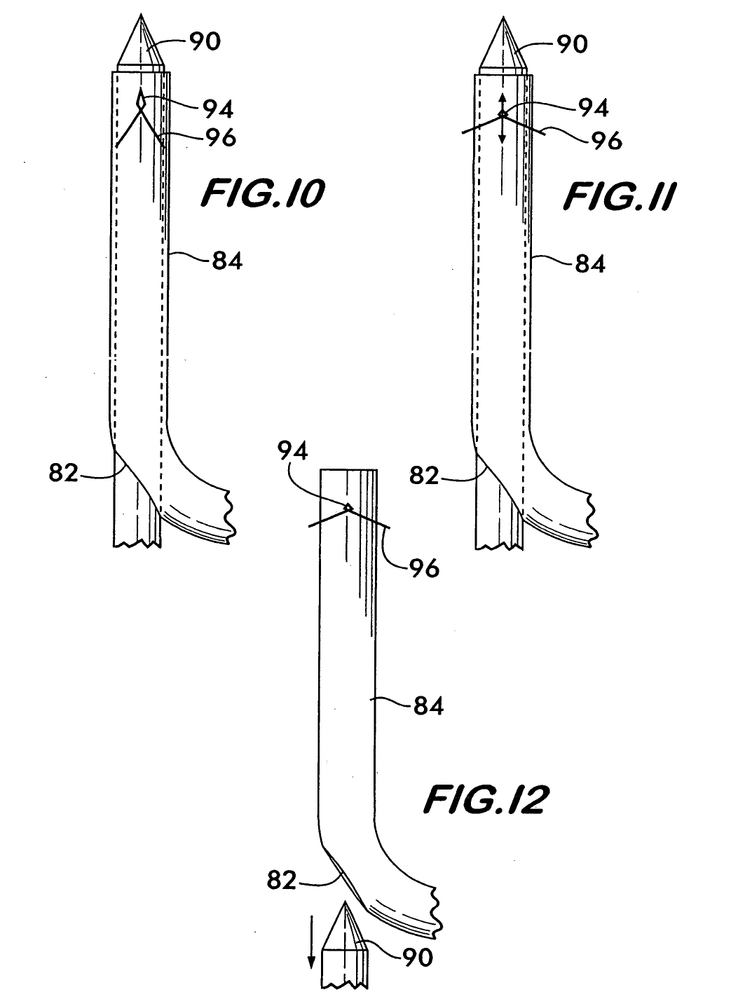

FIG. 9 shows another embodiment of a sling 80 according to the invention. Sling 80 is similar to sling 30 but has openings 82 positioned between the end portions 84 and 86 and the intermediate portion 88. The openings 82 allow tunneling needles 90 to be positioned within the end portions for implantation into the abdominal tissue. Tunneling needles 90 are attached to a tool 92 which facilitates manual manipulation of the needles and also allows for the deployment of mechanical anchors 94 as shown in FIGS. 10-12.

The needles 90 are inserted into the vaginal incision as described previously and through the abdominal tissue. The mechanical anchors 94, in the form of a type of staple having deformable legs 96, are attached to the end portions 84 and 86 but are deployable into engagement with the abdominal tissue by a mechanism (not shown) associated with the tools 92. After the sling 80 is positioned as desired (FIG. 10) the legs 96 of the anchors 94 are deployed (FIG. 11) to engage the abdominal tissue and anchor the sling 80 in position. Tunneling needles 90 are then removed (FIG. 12) and the sling 80 remains anchored in place by the engagement between legs 96 and the abdominal tissue.

All of the sling embodiments according to the invention may have radiopaque markers installed to render the sling visible by fluoroscope techniques and thereby facilitate sling positioning within the abdomen. The markers may, for example, be positioned at point locations to indicate boundaries and orientation of the sling, and/or the markers may comprise filamentary members coated with radiopaque material and interlaced with the filamentary members comprising the sling to render the length of the sling visible during implanting.

Slings according to the invention provide numerous advantages over prior art slings including the mitigation or elimination of erosion, the elimination or reduction of recoil effects on urethral pressure as well as providing a sling wherein the pressure on the urethra may be adjusted without the need for invasive surgery. Furthermore, the sling itself, being a tube, provides a guide for the disposition of anchoring systems or other accessories in that the tube interior defines a path through the abdominal tissue.

1. A urinary incontinence sling positionable in an abdomen between a urethra and a vagina to compress the urethra, said sling comprising an elongated tube having opposite end portions and an intermediate portion positioned between said end portions, said intermediate portion being bendable to form a substantially U-shaped cradle positionable adjacent to the urethra, said end portions being positionable so as to extend through the abdomen in a direction away from the vagina, said end portions being in anchoring engagement with living tissue forming said abdomen, said intermediate portion compressing said urethra with a predetermined force.

2. A sling according to claim 1, wherein said tube has a cross sectional shape selected from the group consisting of a circle, an oval and an ellipsoid.

3. A sling according to claim 1, wherein said tube comprises a plurality of interlaced filamentary members.

4. A sling according to claim 3, wherein said filamentary members are interlaced by a technique selected from the group consisting of knitting, weaving and braiding.

5. A sling according to claim 1, wherein said end portions are substantially inextensible.

6. A sling according to claim 1, wherein said end portions have an extensibility between about 10% and about 20% per unit tension load.

7. A sling according to claim 1, wherein said end portions have an extensibility of about 15% per unit tension load.

8. A sling according to claim 1, wherein said end portions have a textured surface adapted to promote ingrowth of living tissue to facilitate anchoring of said end portions within said abdomen.

9. A sling according to claim 3, wherein said end portions comprise material the promotes a healing response in living tissue.

10. A sling according to claim 9, wherein said material comprises a coating applied to said filamentary members forming said end portions.

11. A sling according to claim 5, wherein said intermediate portion is lengthwise elastically extensible.

12. A sling according to claim 1, wherein said intermediate portion has a smooth surface.

13. A sling according to claim 1, further comprising an elastically expandable and contractible body positioned within said intermediate portion of said tube, said body having a predetermined internal pressure, said pressure substantially determining the compressive force exerted on said urethra by said tube.

14. A sling according to claim 13, wherein said body comprises a fluid-filled balloon.

15. A sling according to claim 13, further comprising a pouch positioned at said intermediate portion of said tube, said pouch being defined by closing said tube at two locations in spaced apart relation to one another, said pouch containing said expandable and contractable body within said intermediate portion.

16. A sling according to claim 1, further comprising an anchor mounted on at least one of said end portions, said anchor being engageable with tissue of said abdomen and adapted to fix the position of said tube therein.

17. A sling according to claim 1, further comprising a needle attached to one of said end portions, said needle adapted to penetrate said tissue and facilitate drawing of said tube through said abdomen.

18. A urinary incontinence sling positionable in an abdomen between a urethra and a vagina to compress the urethra, said sling comprising elongated end portions oppositely disposed and an intermediate portion positioned between said end portions, said intermediate portion being bendable to form a substantially U-shaped cradle positionable adjacent to the urethra, said end portions being substantially inextensible, said end portions being positionable so as to extend through the abdomen in a direction away from the vagina, said end portions being in anchoring engagement with living tissue forming said abdomen, said intermediate portion compressing said urethra with a predetermined force.

19. A sling according to claim 18, wherein said end portions have an extensibility between about 10% and about 20% per unit tension load.

20. A sling according to claim 18, wherein said end portions have an extensibility of about 15% per unit tension load.

21. A sling according to claim 18, wherein said end portions have a textured surface adapted to promote ingrowth of living tissue to facilitate anchoring of said end portions within said abdomen.

22. A sling according to claim 18, wherein said end portions comprise material that promotes a healing response in living tissue.

23. A sling according to claim 22, wherein said material comprises a coating applied to said end portions.

24. A sling according to claim 18, wherein said intermediate portion is lengthwise elastically extensible.

25. A sling according to claim 18, wherein said end portions and said intermediate portion comprise a tube.

26. A sling according to claim 18, wherein said end portions comprise elongated straps.

27. A sling according to claim 26, wherein said intermediate portion comprises a tube.

28. A sling according to claim 26, further comprising an elastically expandable and contractible body positioned within said intermediate portion, said body having a predetermined internal pressure, said pressure substantially determining the compressive force exerted on said urethra by said intermediate portion.

29. A sling according to claim 28, wherein said body comprises a fluid-filled balloon.

30. A sling according to claim 18, futher comprising an anchor mounted on at least one of said end portions, said anchor being engageable with tissue of said abdomen and adapted to fix the position of said end portion therein.

31. A sling according to claim 18, further comprising a needle attached to one of said end portions, said needle adapted to penetrate said tissue and facilitate drawing of said end portion through said abdomen.

32. A sling according to claim 18, wherein said end portions and said intermediate portion comprises interlaced filamentary members.
